(19) United States
(12) Patent Application Publication
(10) Pub. No.: US 2020/0180803 A1
(43) Pub. Date: Jun. 11, 2020

(54) MACHINE AND PROCESS FOR PREPARING STERILIZED FLEXIBLE-BAGS FOR PACKAGING PRODUCTS

(71) Applicant: Liqui-Box Spain SL, Madrid (ES)

(72) Inventor: Jose Vicente MORALES VIZCAINO, Madrid (ES)

(21) Appl. No.: 16/635,357

(22) PCT Filed: Jul. 31, 2018
(86) PCT No.: PCT/IB2018/055743

§ 371 Date: Jan. 30, 2020

(30) Foreign Application Priority Data
Jul. 31, 2017 (ES) U201730918

(51) Int. Cl. B65B 55/08; A61L 2/10; B65B 9/10

(52) U.S. Cl. CPC B65B 55/08; A61L 2/10; A61L 2202/23; A61L 2202/181; B65B 9/10

(57) ABSTRACT

The present disclosure relates to a machine and process for in-line sterilization and making of flexible-bags used for packaging flowable and non-flowable materials. The sterilization is incorporated at one or more stages during the process of making the flexible-bag and packaging the product. Stated another way, the present disclosure relates to a process and machine for sterilizing flexible-bag before and/or after the packaging of the product.

### CROSS REFERENCE TO RELATED APPLICATIONS

This application claims the benefit of Spanish Utility Model Application No. U201730918, filed Jul. 31, 2017, the entirety of which is incorporated herein for any and all purposes.

### TECHNICAL FIELD

The present disclosure relates to a machine and process for in-line sterilization and making of flexible-bags used for packaging flowable and non-flowable materials. The sterilization is incorporated at one or more stages during the process of making the flexible-bag and packaging the product. Stated another way, the present disclosure relates to a process and machine for sterilizing a flexible-bag before and/or after the packaging of the product.

### BACKGROUND

At present, the manufacturers of flexible-bags for fluid, semi-liquid, or liquid, that is flowable materials, for preparing commercially sterile packages often rely on gamma irradiation and accelerated electron beam technology techniques for sterilization. Existing techniques for achieving sterilization or sanitization use the gray unit to measure the ionizing radiation absorbed by a material. The gray (symbol: Gy) is a derived unit of ionizing radiation dose in the International System of Units (SI). A gray is equivalent to the absorption of a joule of ionizing energy per kilogram of irradiated material. A minimum dose of 10 kilograys (kGy) is necessary to be acceptable for the aseptic market for products with a high acid content: for example, tomato, oranges, cider, lemon, grapefruit, prunes, etc. Similarly, a 30 kGy dose is often the minimum necessary for products with low acid content: for example, banana, guava, coconut, water, dairy, etc. However, doses greater than 30 kGy can lead to a cross-linking effect or splitting or excision of the macro-molecular chains in the bag or the food. The aseptic market accepts the values of 10 kGy to 30 kGy.

Existing sterilization techniques warrant a high general operating cost and a difficulty in timely delivery to the client. Moreover, they fail to destroy certain microorganisms, that is, they do not guarantee a 99.9999% sterilization. As a result, the supplier too, will not vouch for the integrity of the product. In addition, the likely change to the molecular chains of the flexible-bag material subjected to the exiting sterilization process restricts the breadth of polymeric materials that can be used for preparing the flexible-bags.

### THE FFS PROCESS

A convenient method of packaging flowable products in thermoplastic film is generally known as the form/fill/seal process. For example, the process is known to those of skill in the art, and described, for example, in U.S. Pat. No. 4,506,494 (Shimoyama, et al.); U.S. Pat. No. 4,589,247 (Tsuruta, et al); U.S. Pat. No. 4,656,818 (Shimoyama, et al.); U.S. Pat. No. 4,768,411 (Su); U.S. Pat. No. 4,808,010 (Vogan); and U.S. Pat. No. 5,467,581 (Everette); all incorporated herein by reference in their entirety. Many horizontal form/fill/seal (HFFS) and vertical form/fill/seal (VFFS) systems are commercially available from manufacturers or suppliers such as Hayssen, Omori, Lapak, and Kartridge Pak. VFFS packaging systems have proven to be very useful in packaging a wide variety of flowable products. An example of such systems is the ONPACK™ flowable food packaging system marketed by Cryovac/Sealed Air Corporation.

Flexible-bags are generally made of polyolefin films, which are used to package flowable products. Examples of such films can be found in U.S. Pat. Nos. 4,503,102; 4,521,437; 5,206,075; 5,364,486; 5,508,051; 5,721,025; 5,879,768; 5,942,579; 5,972,443; 6,117,465; 6,256,966; 6,406,765; 6,416,833; and 6,767,599. All of these patents describe various polymers and blends that are used to manufacture flexible packages such as those described herein. The disclosures of all of these documents are incorporated herein by reference.

The polyolefin film must show high flex-crack resistance and high thermal resistance to maintain the flexible-bag's aseptic characteristic. The aseptic characteristic is engendered, for example, through steam sterilization, which can sometimes result in poor aesthetics and bag properties. Steam sterilization can result in wrinkling of the flexible-bag film, which can often be accompanied by the inner and outer plies of a multiply bag sticking together, or even a bag or pouch made from a monolayer film sticking to itself. Films with inadequate thermal resistance may stretch and deform unacceptably in close proximity to heated machine parts such as sealing jaws found in form, fill and seal machines. The stretched or deformed area of the film may become the weak point of the pouch or bag, at which it will fail prematurely in subsequent shipping and handling.

Many a times, the bag or pouch passes through a long heating tunnel for several minutes which is maintained at roughly the same temperature as the product fill temperature to keep the contents hot and to kill mold and bacteria. The bag or pouch subsequently enters and passes through a long cooling tunnel to cool down to almost room temperature. Films used in such bags or pouches require good thermal resistance so that the films and the seals maintain their integrity while in contact with the hot product. Thus, sterilization is an important step in flexible-bag making and packaging with flowable products. However, the sterilization step is discrete, that is, non-continuous, in which case, it reduces productivity per unit time, or it is harsh, which adversely influences the integrity, and thus the shelf life, of flexible-bag, with or without the product.

In all of these processes, however, sterilization is a separate step. The present invention relates to a process in which the step for sterilizing the flexible bag being made or the flexible bag filled with the flowable product is effected in-line during the continuous manufacturing process, such as the VFFS process, described above. Stated differently, an object of the present disclosure is to develop a machine that not only carries out the process of manufacturing flexible-bags for the packaging of flowable products, for example, fresh food products, but also performs the sterilization process during the flexible-bag making and packaging process.

### SUMMARY OF THE INVENTION

In one aspect, this invention relates to a method of making a sterilized flexible-bag for receiving, storing, and dispensing a product, the method comprising the steps of:
(I) providing a flexible-bag-making machine;
(II) providing at least one UVG radiation sterilization equipment in-line with said flexible-bag-making machine;
(III) optionally providing at least one UVG radiation detection meter;
(IV) making said flexible-bag in said flexible-bag-making machine;
(V) sterilizing said flexible-bag by:
   (i) projecting a UVG radiation onto the polymeric film from which said flexible-bag is made, prior to formation of said flexible-bag, and/or
   (ii) projecting a second UVG radiation onto the partially-made flexible-bag during formation of said flexible-bag, and/or
   (iii) projecting a third UVG radiation on to said flexible bag after formation of said flexible-bag; and
(VI) optionally measuring at least one of the UVG radiation from said at least one UVG radiation sterilization equipment.

In another aspect, this invention relates to a method as recited above, wherein said product is a flowable product.

In yet another aspect, this invention relates to a method of making a sterilized flexible-bag and packaging flowable product in it, the method comprising the steps of:
(I) providing a flexible-bag-making machine;
(II) providing at least one UVG radiation sterilization equipment in-line with said flexible-bag-making machine;
(III) optionally providing at least one UVG radiation detection meter;
(IV) making said flexible-bag and filling said flexible-bag with flowable product in said flexible-bag-making machine;
(V) sterilizing said flexible-bag by:
   (i) projecting a UVG radiation onto the polymeric film from which said flexible-bag is made, prior to formation of said flexible-bag, and/or
   (ii) projecting a second UVG radiation onto the partially-made flexible-bag during formation of said flexible-bag but before filling said flexible-bag with flowable product, and/or
   (iii) projecting a third UVG radiation onto the partially-made flexible-bag during formation of said flexible-bag but after filling said flexible-bag with said flowable product, but before completely sealing said flexible-bag, and/or
   (iii) projecting a fourth UVG radiation on to said flexible bag after formation of said flexible-bag; and
(VI) optionally measuring at least one of the UVG radiation from said at least one UVG radiation sterilization equipment.

In one aspect, this invention relates to a method as recited above, wherein said flexible-bag-making machine is a form/fill/seal machine. In one embodiment, this invention relates to a method as recited above, wherein said for/fill/seal machine is a VFFS machine. In another aspect, this invention relates to methods as recited above wherein said UVG radiation sterilization equipment is an ultraviolet radiation emitting lamp, which is disposed adjacent a sealing unit of said FFS machine. In another embodiment, this invention relates to methods recited above, wherein said UVG sterilization equipment emits UVG radiation in the range of from about 40,000 to 100,000 μW/cm². In yet another embodiment, this invention relates to methods as recited above, wherein said UVG sterilization provides reduction on microbial CFU in the range of 1-6 logarithmic units.

In one embodiment, this invention relates to a machine for making a sterilized flexible-bag for receiving, storing, and dispensing a product, the machine comprising:
(I) a flexible-bag-making machine;
(II) at least one UVG radiation sterilization equipment placed in-line with said flexible-bag-making machine; and
(III) optionally at least one UVG radiation detection meter.

In another embodiment, this invention relates to the machine described above, wherein said product is a flowable product.

In yet another embodiment, this invention relates to the machine described above, wherein said flexible-bag-making machine is a form/fill/seal machine. In yet another embodiment, this invention relates to the machine described above, wherein said for/fill/seal machine is a VFFS machine.

In one embodiment, this invention relates to the machines described above, wherein said UVG radiation sterilization equipment is an ultraviolet radiation emitting lamp, which is disposed adjacent a sealing unit of said FFS machine.

In one embodiment, this invention relates to a sterilized flexible-bag made by methods described above.

In another embodiment, this invention relates to sterilized flexible-bag as recited in above, further comprising a product selected from fruits, vegetables, meats, chips, snacks, and grains. In yet another embodiment, this invention relates to a sterilized flexible-bag comprising product inside said sterilized flexible-bag, made by a method as recited above. In a further embodiment, this invention relates to the sterilized flexible-bag as recited above, wherein said product is a fluid with or without particulates; a liquid with or without particulates; a semi-liquid with or without particulates; a paste with or without particulates; emulsions with or without particulates; preserves with or without particulates; jelly with or without particulates; doughs with or without particulates; ground meat with or without particulates; powders; granular solids; puree with or without particulates; concentrate with or without particulates; mixes with or without particulates; carbonated beverages; non-carbonated beverages; alcoholic liquids; and combinations thereof.

In another embodiment, this invention relates to the sterilized flexible-bag as recited above, wherein said product is selected from milk, water, juice, fruit juice, vegetable juice, crushed fruits, blended fruits, crushed vegetables, blended vegetables, smoothies, oil, ice cream mix, soft margarine, shampoo, liquid soap, detergent, meat paste, cheese, sauce, peanut butter, jam, pie filling, marmalade, sausage meat, gelatin powders, detergents, nuts, sugar, and salt.

In one aspect, the object of the present invention is a sterilizing machine for bags containing liquid or food fluids by ultraviolet germicidal (UVG) radiation. According to an embodiment of the present disclosure, a sterilizing machine for flexible-bags includes a manufacturing sheet of the bags; an ultraviolet radiation emitting lamp disposed such that that the ultraviolet radiation emitting lamp covers the entire width of the manufacturing sheet of the flexible-bags; and a radiation meter. The ultraviolet radiation emitting lamp is disposed adjacent a sealing unit of the sterilizing machine.

According to another embodiment, a method of manufacturing flexible-bag includes the steps of introducing a first sheet of material, for example, a polymeric film, into a flexible-bag-making machine, introducing a second sheet of material into the flexible-bag-making machine, adhering the first sheet of material with the second sheet of material along at least one edge such that a cavity is formed between the first sheet and the second sheet, the cavity being configured to receive a fluid, and projecting ultraviolet radiation onto the first sheet and the second sheet. The projecting step is done prior to the adhering step and is performed by an ultraviolet radiation-emitting lamp disposed on the flexible-bag-making machine.

According to another embodiment, a bag for receiving and storing fluid is formed from a material that has been subject to ultraviolet radiation, such that the bag is sterile. The ultraviolet radiation is supplied by an ultraviolet radiation-emitting lamp.

### BRIEF DESCRIPTION OF THE DRAWINGS

The present application is further understood when read in conjunction with the appended drawings. To illustrate the subject matter, there are shown in the drawings exemplary embodiments of the subject matter; however, the presently disclosed subject matter is not limited to the specific methods, devices, and systems disclosed. In the drawings:

FIG. 1 illustrates a representation of part of the sterilizing machine for the manufacture of liquid-containing bags according to an embodiment; and

FIG. 2 illustrates an internal cross-sectional view of a sterilizing machine according to an embodiment.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

Ranges are used herein in shorthand, to avoid having to list and describe each value within the range. For example, any appropriate value within the range can be selected as the upper value, lower value, or the terminus of the range. As used herein, the singular form of a word includes the plural, and vice versa, unless the context clearly dictates otherwise. Thus, the references “a,” “an,” and “the” are generally inclusive of the plurals of the respective terms. For example, reference to “a method” includes a plurality of such “methods.” Likewise, the terms “include,” “including,” and “or” should all be construed to be inclusive, unless such a construction is clearly prohibited from the context. Similarly, the term “examples,” particularly when followed by a listing of terms, is merely exemplary or illustrative and should not be deemed exclusive or comprehensive.

The term “comprising” is intended to include embodiments encompassed by the terms “consisting essentially of” and “consisting of.” Similarly, the term “consisting essentially of” is intended to include embodiments encompassed by the term “consisting of.” The methods and compositions and other advances disclosed herein are not limited to particular equipment or processes described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

This invention relates to flowable products and non-flowable products.

As used herein, the term “flowable product” encompasses materials that are flowable under gravity or may be pumped. Flowable product includes a fluid, a liquid, a semi-liquid, a paste, and a combination thereof, with or without particulates in them. Flowable product includes food and non-food products. Such materials include liquids, for example, milk, water, juice, fruit juice, oil; emulsions, for example, ice cream mix, soft margarine, shampoo, liquid soap and detergent; pastes, for example, meat pastes, cheese, sauce, and peanut butter; preserves, for example, jams, pie fillings, and marmalade; jellies; doughs; ground meat, for example, sausage meat; powders, for example, gelatin powders, detergents; granular solids, for example, nuts, sugar, and salt; puree; concentrates; mixes; and such materials. The invention described herein is particularly useful for flowable foods. By non-flowable products is meant, generally larger solids, for example solids that are not considered particles or particulate matter. While this invention applies both to flowable products and non-flowable products, and to food products and non-food products, the invention is discussed in terms of flowable products. The discussion applies to non-flowable products as well.

The present invention relates to an in-line sterilization for flexible-bags containing or not containing flowable product such as liquid or food, comprising providing an in-line ultraviolet germicidal (UVG) radiation, which sterilizes at one or more stages of the flexible bag-making and packaging process. This invention also relates to a machine that provides an in-line sterilization for such flexible-bags using UVG radiation.

In one embodiment, the present invention relates to a machine for sterilizing, bag-making, and packaging flexible-bags not comprising a flowable product. In another embodiment, the present invention relates to a machine for sterilizing, bag-making, and packaging flexible-bags comprising a flowable product. The UVG radiation is provided by an emitting lamp disposed such that that the ultraviolet radiation emitting lamp covers the entire width of the film used for making the bag, and a radiation meter. In one embodiment, the ultraviolet radiation emitting lamp is disposed adjacent a sealing unit of the machine.

According to another embodiment, a method of manufacturing a fluid-containing bag includes the steps of introducing a first sheet of polymeric film material into a bag manufacturing machine, introducing a second sheet of polymeric film material into the bag manufacturing machine, adhering the first sheet of material with the second sheet of material along at least one edge such that a cavity is formed between the first sheet and the second sheet, the cavity being configured to receive a flowable product, and projecting ultraviolet radiation onto the first sheet and the second sheet. The projecting step is done prior to the adhering step and is performed by an ultraviolet radiation-emitting lamp disposed on the bag-manufacturing machine.

In one embodiment the bag-making machine is an VFFS or an HFFS machine. In this embodiment, the UVG radiation is projected before the first hot-seal formation for a particular bag. A typical FFS process is described below. In such a process, initially, a lay-flat thermoplastic film is advanced over a forming device to form a tube, on which, then, a longitudinal-fin (vertical) or lap-seal is made, forming a tube. Then, an end-seal is made by transversely sealing across the tube with heated seal-bars to form a conveniently wide, first heat-seal, consequently producing a bag or pouch ready to receive a flowable product. The formed tubular film, i.e., the precursor to the flexible-bag or pouch, is filled with the flowable product, for example, through a central, vertical fill tube.

In the next step, squeeze rollers, spaced apart and above the first heat-seal, squeeze the filled tube and pinch the walls of the flattened tube together. When a length of tubing of the desired height of the bag has been fed through the squeeze rollers, a second heat-seal is made by heat-seal bars, transversely, across the width of the flattened tubing in a relatively wide band, thereby clamping and sealing the film of the tube therebetween. Then, the seal-bars are withdrawn, and the film moves downwardly to be contacted by cooled clamping and severing bars, which clamp the film therebetween. The clamping and severing bars are also provided with a cutting knife to sever the sealed film transversely at about the midpoint of the second heat-seal so that approximately half of the second heat-seal will be on the upper part of a tube, or the next bag to-be-filled, and the other half on the lower tube, or the filled bag.

Thus, one wide-band seal serves as the bottom-seal for one bag and the top-seal for the subsequent bag. When the sealing and severing operation is complete, the squeeze rollers are separated to allow a new charge of product to enter the flattened tube after which the aforementioned described process is repeated thus continuously producing vertical form/fill/seal pouches, which have a bottom end and top end heat seal closure. The process can be a two-stage process where the creation of a transverse heat seal occurs at one stage in the process, and then, downstream of the first stage, a separate pair of cooling/clamping means contact the just-formed transverse heat seal to cool and thus strengthen the seal. In some VFFS processes, an upper transverse seal of a first pouch, and the lower transverse seal of a following pouch, are made, and the pouches cut and thereby separated between two portions of the transverse seals, without the need for a separate step to clamp, cool, and cut the seals.

In one embodiment, the UVG radiation is projected on the lay-flat thermoplastic film, prior to the formation of the longitudinal-fin or lap-seal. In another embodiment, the UVG radiation is projected on the thermoplastic film after it has been formed into a tube, that is, after the formation of the longitudinal-fin or lap-seal.

In yet another embodiment, the UVG radiation is projected on to the thermoplastic film, after the first transverse hot-seal. In another embodiment, the UVG radiation is projected on to the flexible-bag after the filling of flowable product into the flexible-bag and after the formation of the second transverse hot-seal that is on the flexible bag packaged with the flowable product.

In yet another embodiment, the UVG radiation is projected on the lay-flat thermoplastic film, prior to the formation of the longitudinal-fin or lap-seal; and/or the UVG radiation is projected on the thermoplastic film after it has been formed into a tube, that is, after the formation of the longitudinal-fin or lap-seal; and/or the UVG radiation is projected on to the thermoplastic film, after the first transverse hot-seal; and/or the UVG radiation is projected on to the flexible-bag after the filling of flowable product into the flexible-bag and after the formation of the second transverse hot-seal. In one embodiment, the ultraviolet radiation is supplied by an ultraviolet radiation-emitting lamp.

Ultraviolet germicidal radiation (UVG) is a disinfection method that uses wavelengths shorter than visible light. Ultraviolet radiation effectively kills or inactivates microorganisms by destroying nucleic acids and altering their DNA, leaving them unable to perform vital cellular functions. UVG is used in a variety of applications, such as food, air, and water purification. UVG is scientifically proven to interrupt the DNA and RNA structure of the virus, bacteria, molds, and yeast being approved for use by the Food and Drug Administration (FDA) and the United States Department of Agriculture (USDA) in food facilities for surface decontamination.

UVG radiation sterilization of the present invention is less expensive an operation compared to Gamma/E-Beam radiation systems. UVG radiation sterilization of the present invention allows for in-line application during the manufacture of the flexible-bags for flowable products. In addition, the effectiveness of the rays is controlled through the in-line radiometer. The UVG radiation sterilization of the present invention does not produce ozone or secondary contaminants that can migrate to food and affect smell, taste, and safety. The in-line aspect of the sterilization allows for the mitigation of any delay in delivery to the customer because the distribution logistics are optimized. In addition, the UVG radiation sterilization of the present invention does not affect the integrity and functionality of the polymer. It should be noted that the UVG radiation sterilization of the present invention is an environmentally friendly technique. It should also be noted that the in-line aspect of the UVG radiation sterilization of the present invention does not impeded continuity of the manufacture of the flexible-bags and their packaging with the flowable products.

Successful tests have been carried out at a high production cycle, for example, 30 cycles per min. In a preferred embodiment, production cycle in the present invention can be any one of the numbers from the following numbers (in cycles/min), or in a range defined by any two numbers (including the endpoints) from the following numbers. 1, 2, 3, 4, 5, 6, 7, 8, 9, 10, 11, 12, 13, 14, 15, 16, 17, 18, 19, 20, 21, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

If a flexible-bag making operation can operate at a production cycle over the one denoted above, the UVG radiation sterilization of the present invention is capable of handling the sterilization without any significant delay in the flexible-bag making operation or an operation for flexible-bag-making and in-line packaging with flowable product.

In one embodiment, a dose of UVG radiation as measured in KμW/cm², that is, kilo-micro-watts/square centimeter or mW/cm², that is, milliwatts/square centimeter, is one of the following numbers, or in a range defined by any one of the numbers: 1, 2, 3, 4, 5, 6, 7, 8, 9, 10, 11, 12, 13, 14, 15, 16, 17, 18, 19, 20, 21, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

For example, a dose of 60,000 μW/cm² or 60 mW reduces at least 4 logarithmic CFUs (same as a 30 kGy dose of Gamma/E-Beam sterilization). In one embodiment, this sterilization also reduces the microbial presence by 1, 2, 3, 4, 5, or 6 or more logarithmic units of CFU (colony-forming-units). A 6 logarithmic unit reduction of CFU would correspond to 99.9999% sterilization.

In one embodiment, the sterilization process is carried out in a non-sterile environment. In another embodiment, the sterilization is carried out in an aseptic environment.

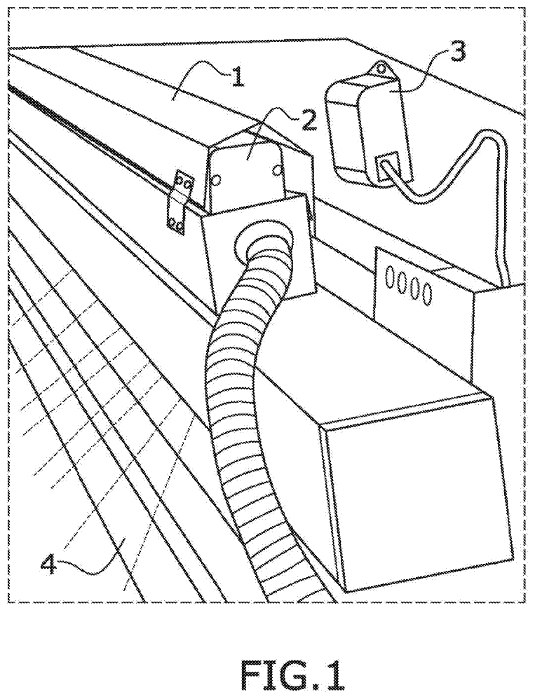

Referring to FIG. 1, a manufacturing machine is shown. An ultraviolet radiation-emitting lamp 2 is disposed on the bag-manufacturing machine. The ultraviolet radiation emitting lamp 2 is disposed adjacent a polymeric film 4 of material. Prior to cutting and gluing of the material, the ultraviolet radiation-emitting lamp 2 may project ultraviolet rays onto the polymeric film 4. The ultraviolet rays are projected onto the side of the polymeric film 4 that is to be sterilized. The ultraviolet rays may cover the entire width of the polymeric film 4.

In some embodiments, the ultraviolet radiation-emitting lamp 2 may be covered by a protective casing 1. The casing offers protection to the ultraviolet radiation-emitting lamp 2. This is advantageous in some embodiments where flexible-bags do not need to be sterilized or when an operator desires to be protected from ultraviolet radiation.

In some embodiments, the sterilizing machine may be designed such that if the protection housing 1 is moved or dislodged unintentionally, the sterilizing machine will stop or pause operation and stop the polymeric film sheet 4 from advancing. In some embodiments, if the radiation-emitting lamp 2 stops emitting ultraviolet radiation, the sterilizing machine may also pause or terminate operation to prevent advancing of the polymeric film 4. This may be advantageous to ensure that all manufactured bags are properly sterilized and that no unsterilized bags are manufactured during this process.

The sterilization machine may include a radiation meter 3 associated with the radiation emitting lamp 2. The radiation meter 3 may detect whether the radiation-emitting lamp 2 is emitting ultraviolet radiation, and, if so, if the proper amount is being emitted. The radiation meter 3 may detect whether too much or not enough ultraviolet radiation is being emitted.

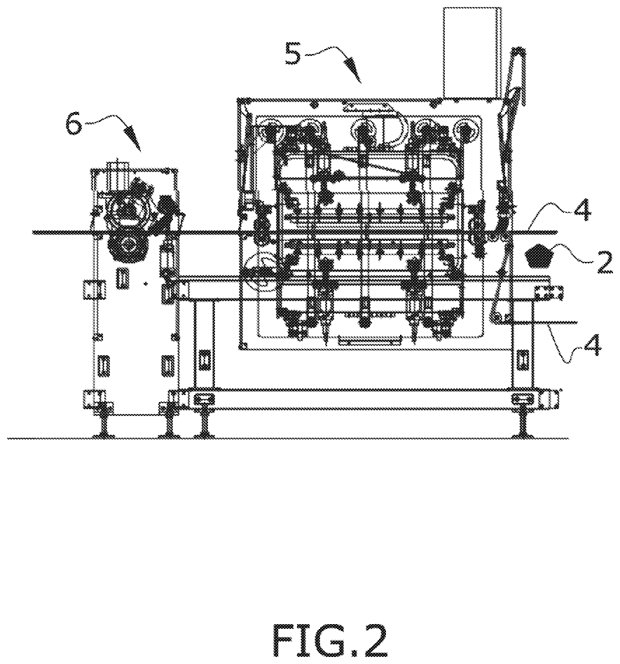

Referring to FIG. 2, the placement of the radiation-emitting lamp 2 is shown according to an embodiment. The radiation-emitting lamp 2 may be disposed such that radiation can be projected onto a plurality of polymeric films 4 as the polymeric films 4 move through the polymeric film forming and sterilization machine. As seen in FIG. 2, a first polymeric film 4 may pass on one side of the radiation emitting lamp 4 (e.g., above the lamp 4), while a second sheet 4 may pass on another side of the radiation emitting lamp 4 (e.g., below the lamp 4). The two polymeric films 4 then proceed to be sealed after they had been sterilized with projected ultraviolet rays from the radiation-emitting lamp 4. The sealing may be done with a sealing unit and a traction unit 5.

The sterilized bags may include various food products for storage, transportation, and/or distribution. The bags may be designed to hold even solid food, such as fruits, vegetables, meats, grains, or other food. The sterilized bags may also be configured to receive and store liquids or semi-liquids of varying viscosities, such as carbonated beverages, alcoholic liquids, fruit or vegetable juices, crushed or blended fruits or vegetables (e.g., smoothies), or another liquid or semi-liquid comestible.

In another embodiment, the machine for sterilizing and manufacturing bags as described above further includes a material loading portion that is configured to introduce one or more of the food products described above into a sterilized manufactured bag. In an alternative embodiment, the sterilized manufactured bag may be removed from the machine and then filled with food products at a later time or at another facility.

The sterilized bags containing food products may be designed to be stored in freezers or refrigerators, as well as in warm environments.

1. A method of making a sterilized flexible-bag for receiving, storing, and dispensing a product, the method comprising the steps of:
(I) providing a flexible-bag-making machine;
(II) providing at least one UVG radiation sterilization equipment in-line with said flexible-bag-making machine;
(III) optionally providing at least one UVG radiation detection meter;
(IV) making said flexible-bag in said flexible-bag-making machine;
(V) sterilizing said flexible-bag by:
(i) projecting a UVG radiation onto the polymeric film from which said flexible-bag is made, prior to formation of said flexible-bag, and/or
(ii) projecting a second UVG radiation onto the partially-made flexible-bag during formation of said flexible-bag, and/or
(iii) projecting a third UVG radiation on to said flexible bag after formation of said flexible-bag; and
(VI) optionally measuring at least one of the UVG radiation from said at least one UVG radiation sterilization equipment.

2. The method as recited in claim 1, wherein said product is a flowable product.

3. A method of making a sterilized flexible-bag and packaging flowable product in it, the method comprising the steps of:
(I) providing a flexible-bag-making machine;
(II) providing at least one UVG radiation sterilization equipment in-line with said flexible-bag-making machine;
(III) optionally providing at least one UVG radiation detection meter;
(IV) making said flexible-bag and filling said flexible-bag with flowable product in said flexible-bag-making machine;
(V) sterilizing said flexible-bag by:
(i) projecting a UVG radiation onto the polymeric film from which said flexible-bag is made, prior to formation of said flexible-bag, and/or
(ii) projecting a second UVG radiation onto the partially-made flexible-bag during formation of said flexible-bag but before filling said flexible-bag with flowable product, and/or
(iii) projecting a third UVG radiation onto the partially-made flexible-bag during formation of said flexible-bag but after filling said flexible-bag with said flowable product, but before completely sealing said flexible-bag, and/or
(iii) projecting a fourth UVG radiation on to said flexible bag after formation of said flexible-bag; and
(VI) optionally measuring at least one of the UVG radiation from said at least one UVG radiation sterilization equipment.

4. The method as recited in claim 2, wherein said flexible-bag-making machine is a form/fill/seal machine.

5. The method as recited in claim 4, wherein said for/fill/seal machine is a VFFS machine.

6. The method as recited in claim 1, wherein said UVG radiation sterilization equipment is an ultraviolet radiation emitting lamp, which is disposed adjacent a sealing unit of said FFS machine.

7. The method as recited in claim 1, wherein said UVG sterilization equipment emits UVG radiation in the range of from about 40,000 to 100,000 μW/cm².

8. The method as recited in claim 1, wherein said UVG sterilization provides reduction on microbial CFU in the range of 1-6 logarithmic units.

9. A machine for making a sterilized flexible-bag for receiving, storing, and dispensing a product, the machine comprising:
a flexible-bag-making machine;
(II) at least one UVG radiation sterilization equipment placed in-line with said flexible-bag-making machine; and
(III) optionally at least one UVG radiation detection meter.

10. The machine as recited in claim 9, wherein said product is a flowable product.

11. The machine as recited in claim 10, wherein said flexible-bag-making machine is a form/fill/seal machine.

12. The machine as recited in claim 11, wherein said for/fill/seal machine is a VFFS machine.

13. The machine as recited in claim 9, wherein said UVG radiation sterilization equipment is an ultraviolet radiation emitting lamp, which is disposed adjacent a sealing unit of said FFS machine.

14. A sterilized flexible-bag made by the method recited in claim 1.

15. The sterilized flexible-bag as recited in claim 14, further comprising a product selected from fruits, vegetables, meats, chips, snacks, and grains.

16. A sterilized flexible-bag comprising product inside said sterilized flexible-bag, made by a method as recited in claim 3.

17. The sterilized flexible-bag as recited in claim 16, wherein said product is a fluid with or without particulates; a liquid with or without particulates; a semi-liquid with or without particulates; a paste with or without particulates; emulsions with or without particulates; preserves with or without particulates; jelly with or without particulates; doughs with or without particulates; ground meat with or without particulates; powders; granular solids; puree with or without particulates; concentrate with or without particulates; mixes with or without particulates; carbonated beverages; non-carbonated beverages; alcoholic liquids; and combinations thereof.

18. The sterilized flexible-bag as recited in claim 17, wherein said product is selected from milk, water, juice, fruit juice, vegetable juice, crushed fruits, blended fruits, crushed vegetables, blended vegetables, smoothies, oil, ice cream mix, soft margarine, shampoo, liquid soap, detergent, meat paste, cheese, sauce, peanut butter, jam, pie filling, marmalade, sausage meat, gelatin powders, detergents, nuts, sugar, and salt.
